(19) United States
(12) Patent Application Publication
(10) Pub. No.: US 2013/0281878 A1
(43) Pub. Date: Oct. 24, 2013

(54) Fabric Sleeve for Niddah Inspection

(71) Applicant: ELAZAR WINBERG, Jerusalem (IL)

(72) Inventor: ELAZAR WINBERG, Jerusalem (IL)

(21) Appl. No.: 13/858,051

(22) Filed: Apr. 7, 2013

(30) Foreign Application Priority Data
Apr. 19, 2012 (IL) 219280

(51) Int. Cl. A61B 5/00

(52) U.S. Cl. CPC A61B 5/4337
USPC 600/551

(57) ABSTRACT

A fabric sleeve that is worn on the finger and is designed to be used for the Niddah inspection. The sleeve comprises a tubular piece of fabric that is closed at one end and has an opening at the other end into which the user inserts her finger to perform the Niddah inspection.

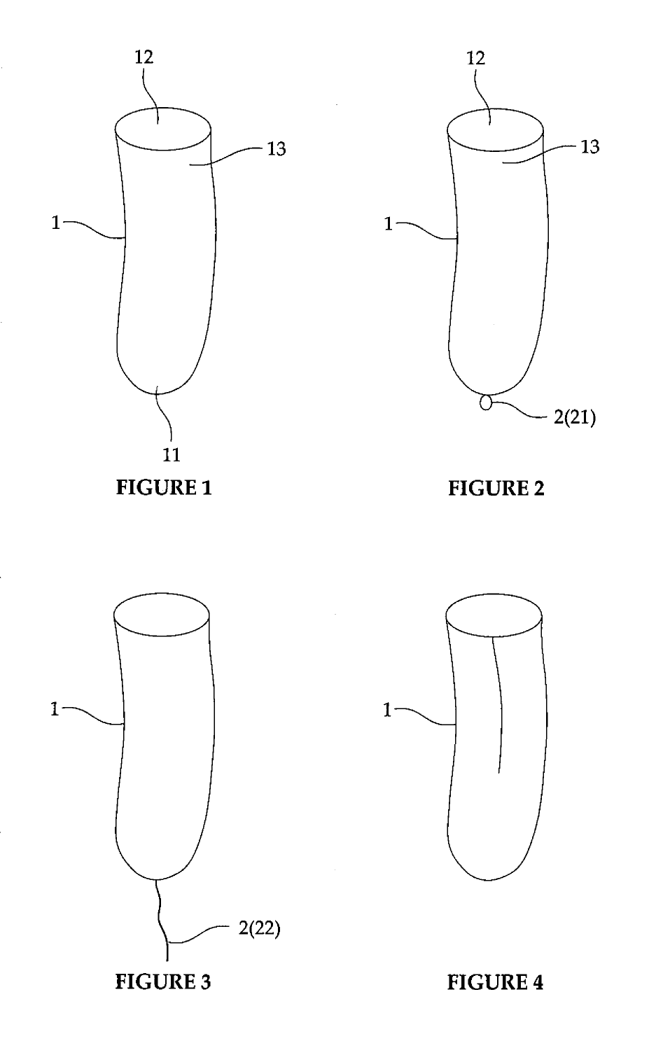

### TECHNICAL FIELD

The present invention refers to a fabric sleeve for inspecting Niddah according to the Halakhah (Jewish rabbinical law).

### BACKGROUND ART

According to the laws of family purity, which are part of the Jewish Halakhah (religious laws), a woman may engage in intercourse only after seven “clean” days have passed since the end of her monthly period. Thus, a Kosher, Halakhah-observant woman must conduct internal inspections every day to see whether there is any blood in her vagina (hereinafter “the inspection” or “the Niddah inspection”). The Niddah inspection is customarily performed using a piece of fabric that the woman wraps around her finger. When in doubt, the woman must show the piece of fabric to a rabbi so that he can determine whether the staining on the fabric is blood or other secretions. It is very important to keep the fabric in the same condition it was in when it was removed from the vagina so as not to disturb the findings. Since the inspection is performed using a piece of fabric that is usually rectangular in shape and is wrapped around the finger, creases and folds often form in the fabric, making it difficult to determine the nature of the findings. Sometimes such creases and folds even accidentally cause internal injury or scratching in women with sensitive skin. Needless to say, it is also quite inconvenient to use a piece of fabric to perform the inspection. The present invention offers an efficient and suitable solution to these problems.

### THE DRAWINGS

The drawings attached to the present application are not intended to limit the scope of the invention and the possible ways of its application. The drawings are intended only to illustrate the invention and constitute only one of many possible ways of its application.

Drawing No. 1 depicts the sleeve (1) with a closed bottom (11) and an opening (12) at the top (13).

Drawing No. 2 depicts the sleeve (1) with a removal aid (2) in the form of a protrusion (21).

Drawing No. 2 depicts the sleeve (1) with a removal aid (2) in the form of a piece of thread (22).

Drawing No. 4 depicts the sleeve (1) with a closable opening in closed position.

### The Invention

The main objective of the present invention is to provide a fabric sleeve (1) that is designed to enable observant Jewish women to perform the Niddah inspection. The sleeve (1) is closed at one end and open at the other so that the sleeve may be inserted over a finger in order to perform the inspection. Another objective of the present invention is to provide a sleeve (1) as mentioned whereby its shape enables it to be removed from the finger after the inspection without turning it inside out, i.e. so that the findings remains on the outside of the sleeve since the sleeve is not turned inside out.

The basic design of the sleeve (1) is depicted in Drawing No. 1. The sleeve comprises a tubular piece of fabric that is closed at one end (11) and has an opening (12) at the other end into which the user inserts her finger in order to perform the inspection. The sleeve (1) may be shaped like a finger of a glove and it can be made in a variety of sizes to fit a variety of standard finger sizes, as is customary in the manufacture of standard gloves. The size of the sleeve (1) should be such that it stretches over the finger when it is put on, so that no folds or creases form during use.

Using the sleeve (1): The user inserts her finger into the sleeve (1), performs the internal inspection, extracts the finger along with the sleeve (1), and removes the sleeve (1) from the finger without turning it inside out, so that any findings on the sleeve (1) will remain on the outside and will not be disturbed by turning the sleeve (1) inside out. The importance of being able to remove the sleeve (1) from the finger without turning it inside out is hereby emphasized.

The sleeve (1) can and should be designed so that its top part (13) is relatively wide, so as to facilitate its removal from the finger without turning it inside out. Thus, in order to remove the sleeve (1), the user can simply insert a finger between the sleeve (1) and the finger it is on, and push the sleeve (1) off the finger without turning it inside out.

The sleeve (1) may be equipped with a removal aid (2) to facilitate the removal of the sleeve (1) from the finger without turning it inside out. The removal aid (20 may be a protrusion (21) located on the bottom (11) of the sleeve (1), as depicted for instance in Drawing No. 2. Alternatively, the removal aid (2) may be a piece of string (22) that is attached to the bottom (11) of the sleeve so as to facilitate its removal from the finger, as depicted for instance in Drawing No. 3.

Another possible solution to the aforementioned problem is to design the sleeve (1) so that the opening (13) and may be closed using a weak adhesive and opened upon completion of the inspection. Drawing No. 4 depicts the sleeve (1) with the said opening in closed position.

The sleeve (1), subject of the invention, enables the user to perform the Niddah inspection in an easy, convenient and efficient manner. The part of the sleeve (1) that comes in contact with the inspected area has no creases or folds, thus preventing internal injury and scratches in women with sensitive skin. The sleeve (1) may be very easily removed from the finger without turning it inside out and thus without disturbing the findings.

What is claimed is:

1. A fabric sleeve into which a finger may be inserted, that is designed to be used for the Niddah inspection, and which comprises a tubular piece of fabric that is closed at its bottom and has a top opening into which the finger is inserted.

2. The sleeve mentioned in Claim No. 1 whereby its top part is relatively wide so that it can be removed from the finger relatively easily, without turning it inside out.

3. The sleeve mentioned in Claim No. 1 whereby it is also equipped with a removal aid that is designed to enable relatively easy removal of the sleeve from the finger, without turning it inside out.

4. The sleeve mentioned in Claim No. 3 whereby the removal aid is a protrusion located on the bottom of the sleeve.

5. The sleeve mentioned in Claim No. 3 whereby the removal aid is a piece of string attached to the bottom of the sleeve.

6. The sleeve mentioned in Claim No. 1 whereby its top part is open and can be closed over the finger and opened upon completion of the inspection in a way that enables the user to remove the sleeve from the finger without turning it inside out.
